(19) United States
(12) Patent Application Publication
(10) Pub. No.: US 2020/0289310 A1
(43) Pub. Date: Sep. 17, 2020

(54) INTRAORAL DEVICE AND METHOD OF USING SAME

(71) Applicant: David Cote, Gatineau (CA)

(72) Inventor: David Cote, Gatineau (CA)

(21) Appl. No.: 16/719,606

(22) Filed: Dec. 18, 2019

(30) Foreign Application Priority Data
Mar. 16, 2019 (GB) 1903618.5

(51) Int. Cl. A61F 5/56

(52) U.S. Cl. CPC A61F 5/566

(57) ABSTRACT

An intraoral device having parts thereof that are positionable between the anterior teeth and an oral applicance. The intraoral device is mounted to posterior teeth to reduce or eliminate the pressure exerted on the anterior teeth by the oral appliance.

### FIELD OF THE INVENTION

The present invention relates to the general field of medical devices and is more specifically concerned with intraoral devices and methods of using same.

### BACKGROUND

Oral appliances, such as Mandibular Advancement Devices (MAD), are well known and are particularly useful, for example, in the treatment of snoring and sleep apnea in patients, as well as other types of possible treatment and therapy involving the mandibular arch. Selected models of these devices may comprise portions or elements thereof that are in contact with at least portions or the entire upper and lower dental arches of the patient often overnight, every night, and for relatively long periods of time. Most notably, some models of mandibular advancement devices comprise an upper jaw engaging element configured to apply a rearward force mainly on a transversal anterior surface portion of the upper dental arch of the patient, in cooperation with a lower jaw engaging element configured to simultaneously apply, in a counter action, a forward force mainly on a transversal posterior surface portion of the lower dental arch, so as to bring forward, or advance, the mandibular.

While these devices can generally fulfill the main objective of treating snoring, obstructive sleep apnea and other conditions involving the mandibular, their repeated and prolonged contact with the teeth of the patient may eventually cause dental alignment and/or dental pain issues which, in turn, may require further dental treatment and therapy. Also, for some patients, there is a lack of retention on one or both arches that prevent the use of a conventional appliance.

Against this background, there exists a need in the industry to provide devices and method to mitigate the above-noted disadvantages of oral appliances. An object of the present invention is therefore to provide such devices and methods.

### SUMMARY OF THE INVENTION

In a broad aspect, there is provided an intraoral device for preventing teeth displacement when using a mandibular advancement device, the mandibular advancement device including a maxillary component defining a maxillary component abutment surface for exerting a posterior force against a labial surface of maxillary anterior teeth, a mandibular component defining a mandibular component abutment surface for exerting an anterior force against a lingual surface of mandibular anterior teeth, and a link extending therebetween, the intraoral device comprising: an upper element defining a pair of upper element engaging portions configured for retentive engagement with a respective posterior lateral portion of a maxillary dental arch and an upper element intermediate member extending between the upper element engaging portions; a lower element defining a pair of lower element engaging portions configured for retentive engagement with a respective posterior lateral portion of a mandibular dental arch and a lower element intermediate member extending between the lower element engaging portions. In use, the upper element intermediate member is positionable between the maxillary component abutment surface and the maxillary anterior teeth and the lower element intermediate member is positionable between the mandibular component abutment surface and the mandibular anterior teeth. Forces exerted by the maxillary and mandibular components respectively on maxillary and mandibular dental arches are distributed at least partially to the posterior lateral portions thereof.

There may also be provided an intraoral device wherein the upper element engaging portions each include a respective upper collar configured and sized for encircling one or more respective ones of the maxillary posterior teeth.

There may also be provided an intraoral device wherein the upper element engaging portions each include a respective upper collar configured and sized for encircling a respective one of the maxillary posterior teeth.

There may also be provided an intraoral device wherein the upper element intermediate member includes a susbtantially U-shaped upper wire extending between the upper collars, the upper collars being provided laterally inwardly relative to the upper wire.

There may also be provided an intraoral device further including at least one resiliently deformable upper spacer mounted to the upper wire for spacing apart the upper wire from the maxillary posterior teeth along a posterior portion of the upper wire.

There may also be provided an intraoral device wherein the lower element engaging portions each include a respective lower collar configured and sized for encircling one or more respective ones of the mandibular posterior teeth.

There may also be provided an intraoral device wherein the lower element engaging portions each include a respective lower collar configured and sized for encircling a respective one of the mandibular posterior teeth.

There may also be provided an intraoral device wherein the lower element intermediate member includes a susbtantially U-shaped lower wire extending between the lower collars, the lower collars being provided laterally outwardly relative to the lower wire.

There may also be provided an intraoral device further including at least one resiliently deformable lower spacer mounted to the lower wire for spacing apart the lower wire from the mandibular posterior teeth along a posterior portion of the upper wire.

There may also be provided an intraoral device wherein the upper element engaging portions and the upper element intermediate member are formed integrally in an upper shell defining an upper shell inner surface conforming to at least part of an outer surface of the maxillary dental arch.

There may also be provided an intraoral device wherein the upper shell includes an upper shell body made of a body material and an insert made of an insert material provided inside the upper shell body and defining part of the upper shell inner surface, the insert material being more rigid than the body material.

There may also be provided an intraoral device wherein the body material is a polymer and wherein the upper shell also includes an upper reinforcing wire extending between the upper element engaging portions and along the upper element intermediate member, the upper reinforcing wire being metallic and coupled to the upper shell body for mechanically reinforcing the upper shell body.

There may also be provided an intraoral device wherein the upper reinforcing wire is terminated by a pair of hooks in the upper element engaging portion, the hooks being configured and sized for curving around a respective one of the maxillary posterior teeth.

There may also be provided an intraoral device wherein the upper reinforcing wire extends through the insert.

There may also be provided an intraoral device wherein the upper reinforcing wire is configured to be laterally outward and anterior relative to the maxillary dental arch when the upper shell is operationally mounted to the maxillary dental arch.

There may also be provided an intraoral device wherein the lower element engaging portions and the lower element intermediate member are formed integrally in a lower shell defining a lower shell inner surface conforming to at least part of an outer surface of the mandibular dental arch.

There may also be provided an intraoral device wherein the lower shell includes a lower shell body made of a body material and an insert made of an insert material provided inside the lower shell body and defining part of the lower shell inner surface, the insert material being more rigid than the body material.

There may also be provided an intraoral device wherein the body material is a polymer and wherein the lower shell also includes a lower reinforcing wire extending between the lower element engaging portions and along the lower element intermediate member, the lower reinforcing wire being metallic and coupled to the lower shell body for mechanically reinforcing the lower shell body.

There may also be provided an intraoral device wherein the lower reinforcing wire is terminated by a laterally outwardly extending segment in each one of the lower element engaging portions.

There may also be provided an intraoral device wherein the lower reinforcing wire extends through the insert.

There may also be provided an intraoral device wherein the lower reinforcing wire is configured to be laterally inwardly and posterior relative to the mandibular dental arch when the lower shell is operationally mounted to the mandicular dental arch.

In another broad aspect, there is provided a method for preventing, delaying or reducing teeth displacement in a mouth of an intended user when using a mandibular advancement device, the mandibular advancement device including a maxillary component defining a maxillary component abutment surface, a mandibular component defining a mandibular component abutment surface, and a link extending therebetween, the method comprising: positioning an upper element intermediate member adjacent a labial surface of maxillary anterior teeth; anchoring the upper element intermediate member to one or more maxillary posterior teeth; positioning the maxillary component in the mouth with the maxillary component abutment surface facing the labial surface of the maxillary anterior teeth so that the upper element intermediate member is between at least part of the maxillary component abutment surface and the maxillary anterior teeth; positioning a lower element intermediate member adjacent a lingual surface of mandibular anterior teeth; anchoring the lower element intermediate member to one or more mandibular posterior teeth; positioning the mandibular component in the mouth with the mandibular component abutment surface facing the lingual surface of the mandibular anterior teeth so that the lower element intermediate member is between at least part of the mandibular component abutment surface and the mandibular anterior teeth; biasing the mandibular component in an anterior direction relative to the maxillary component; and distributing at least partially anterior forces exerted by the mandibular component and posterior forces exerted by the maxillary component respectively to the mandibular and maxillary posterior teeth.

There may also be provided a method wherein upper and lower element intermediate members include respectively an upper wire and a lower wire, and wherein positioning the mandibular component in the mouth so that the lower element intermediate member is between at least part of the mandibular component abutment surface and the mandibular anterior teeth includes positioning the mandibular component and the lower wire so that the lower wire is between the mandibular component abutment surface and the mandibular anterior teeth; and positioning the maxillary component in the mouth so that the upper element intermediate member is between at least part of the maxillary component abutment surface and the maxillary anterior teeth includes positioning the maxillary component and the upper wire so that the upper wire between the maxillary component abutment surface and the maxillary anterior teeth.

There may also be provided a method wherein anchoring the upper element intermediate member to one or more maxillary posterior teeth includes inserting a pair of upper collars each extending at a respective end of the upper wire around the one or more of the maxillary posterior teeth located on a respective lateral side on which the upper collars are located; and anchoring the lower element intermediate member to one or more mandibular posterior teeth includes inserting a pair of lower collars each extending at a respective end of the lower wire around the one or more of the mandibular posterior teeth located on a respective lateral side on which the lower collars are located.

There may also be provided a method further comprising spacing apart the upper and lower wires from respectively at least some of the maxillary and mandibular posterior teeth with one or more resiliently deformable spacer positioned between the maxillary and mandibular posterior teeth and respectively the upper and lower wires.

There may also be provided a method comprising covering at least some of the mandibular and maxillary teeth with respectively a lower shell and an upper shell and positioning the maxillary and mandibular components in the mouth over respectively the upper and lower shells.

In another broad aspect, there is provided an intraoral device for preventing teeth displacement when using a mandibular advancement device, the mandibular advancement device including a maxillary component defining a maxillary component abutment surface for exerting a posterior force against a labial surface of maxillary anterior teeth, a mandibular component defining a mandibular component abutment surface for exerting an anterior force against a lingual surface of mandibular anterior teeth, and a link extending therebetween, the intraoral device comprising an engaging portions configured for retentive engagement with a posterior lateral portion of a dental arch and an intermediate member extending from the engaging portion configured for conforming to an anterior portion of the dental arch. In use, the intermediate member is positioned between either the maxillary component abutment surface and the maxillary anterior teeth or between the mandibular component abutment surface and the mandibular anterior teeth. Forces exerted by the maxillary and mandibular components respectively on one of the maxillary and mandibular dental arches are distributed at least partially to the posterior lateral portions thereof.

Advantageously, in some embodiments, the proposed device is relatively inexpensive to manufacture and can prevent, reduce or delay dental alignment problems caused by the use of intraoral devices.

The present application claims priority from UK Request for a Patent 1903618.5 filed Mar. 16, 2019, the contents of which is hereby incorporated by reference in its entirety.

Other objects, advantages and features of the present invention will become more apparent upon reading of the following non-restrictive description of some embodiments thereof, given by way of example only with reference to the accompanying drawings.

### BRIEF DESCRIPTION OF THE DRAWINGS

FIG. 1, in a perspective view, illustrates an embodiment of an upper element part of an intraoral device, according to the present invention, here shown engaged on a maxilliary dental arch;

FIG. 2, in a perspective view, illustrates an embodiment of a lower element part of the intraoral device, according to the present invention, here shown engaged on a mandibular dental arch;

FIG. 3, in a perspective view, illustrates an alternate embodiment of an intraoral device, according to the present invention;

FIG. 4, in a bottom plan view, illustrates the upper element shown in FIG. 1;

FIG. 5, in a top plan view, illustrates the lower element shown in FIG. 1,

FIG. 6, in a cross-sectional side view, illustrates the upper and lower elements in FIGS. 1 and 2, here shown having at least part thereof between the teeth and a mandibular advancement device;

FIG. 7, in a bottom plan view, illustrates an embodiment of an upper element part of an intraoral device, according to an alternative embodiment of the present invention, here shown engaged on a maxilliary dental arch;

FIG. 8, in a perspective view, illustrates an embodiment of a lower element part of the intraoral device partially shown in FIG. 7, here shown engaged on a mandibular dental arch; and

FIG. 9, in a cross-sectional side view, illustrates the upper and lower elements in FIGS. 7 and 8, here shown having at least part thereof between the teeth and a mandibular advancement device;

### DETAILED DESCRIPTION

The terms “substantially” and “about” are used throughout this document to indicate variations in the thus qualified terms. These variations are variations that do not materially affect the manner in which the invention works and can be due, for example, to uncertainty in manufacturing processes or to small deviations from a nominal value or ideal shape that do not cause significant changes to the invention.

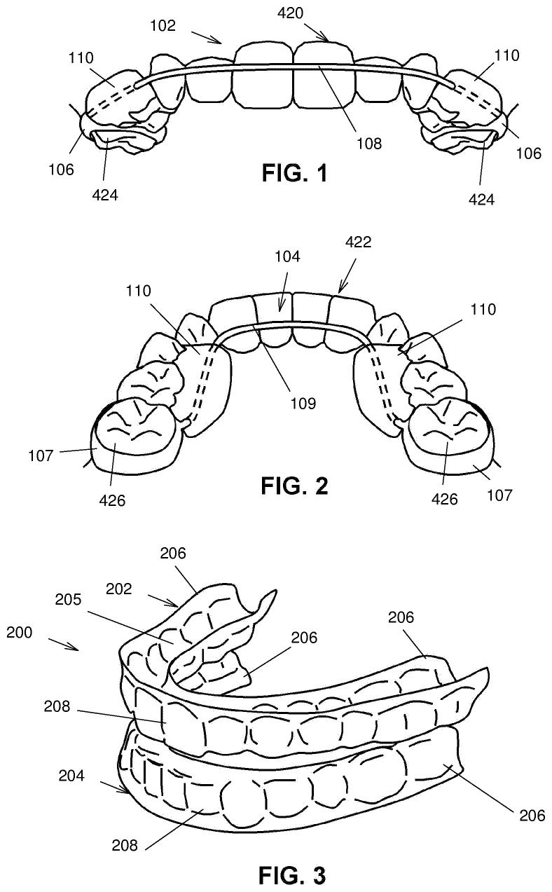

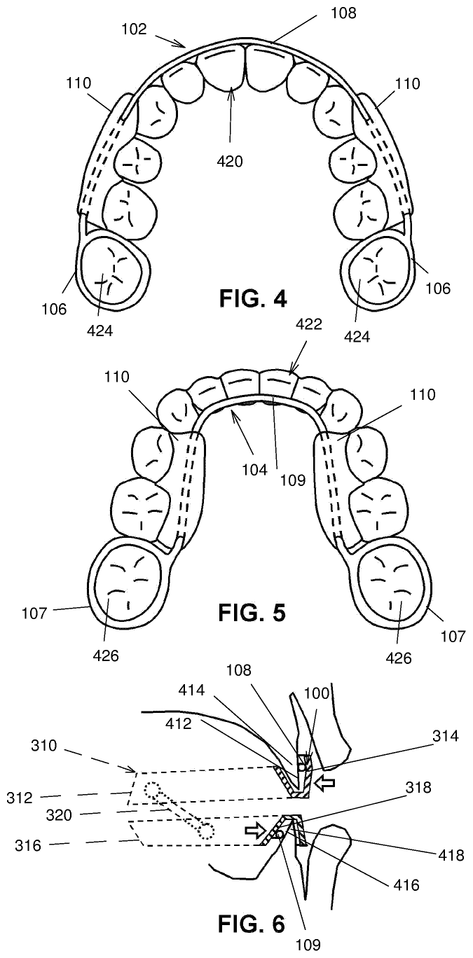

FIGS. 1, 2, and 4 to 6 inclusively, illustrate various aspects of an embodiment, according to the present invention, of an intraoral device 100. The intraoral device 100 is usable for preventing, reducing or delaying the occurrence of dental issues, such as teeth displacement, in patients using a mandibular advancement device 310 or the like, such as the one illustrated in FIG. 6. For example, the proposed intraoral device 100 is usable with mandibular advancement devices 310 manufactured by Panthera™, as shown at www.pantheradental.com or mandibular advancement devices manufactured by Somnodent, as shown at www.Somnodent.com. The mandibular advancement device 310 includes a maxillary component 312 defining a maxillary component abutment surface 314 for exerting a posterior force against a labial surface 412 of 414, a mandibular component 316 defining a mandibular component abutment surface 318 for exerting an anterior force against a lingual surface 416 of mandibular anterior teeth 418, and a link 320 extending therebetween. The link 320 is typically configured for biasing the mandibular component 316 in an anterior direction and biasing the maxillary component 312 in a posterior direction relative to each other. In the absence of the intraoral device 100, the mandibular component abutment surface 318 would abut against the lingual surface 416 of the mandibular anterior teeth 418 and the maxillary component abutment surface 314 would abut against the labial surface 412 of the maxillary anterior teeth 414.

In the presently described embodiment, the intraoral device 100 comprises an upper element 102 and a lower element 104, seen respectively in FIGS. 1 and 2 for example. The upper element 102 defines a pair of upper element engaging portions 106 configured for retentive engagement with a respective posterior lateral portion of a maxillary dental arch 420 and an upper element intermediate member 108 extending between the upper element engaging portions 106. Similarly, the lower element 104 defines a pair of lower element engaging portions 107 configured for retentive engagement with a respective posterior lateral portion of a mandibular dental arch 422 and a lower element intermediate member 109 extending between the lower element engaging portions 107.

In some embodiments, the upper element engaging portions 106 each include a respective upper collar configured and sized for encircling one or more respective ones of the maxillary posterior teeth 424, for example one maxillary posterior tooth 424 each, such as a molar or a premolar. Also, the upper element intermediate member 108 takes the form of a susbtantially U-shaped upper wire extending between the upper collars, the upper collars being provided laterally inwardly relative to the upper wire. Thus, the pair of upper element engaging portions 106 and the upper element intermediate member 108 cooperatively form a substantially U-shaped configuration. In some embodiments of the invention, each upper collar is made of a metal conventionally used in dental treatment and therapy.

Similarly, the lower element engaging portions 107 each include a respective lower collar configured and sized for encircling one or more respective ones of the mandibular posterior teeth 426, for example one maxillary posterior tooth 426 each, such as a molar or a premolar. Also the lower element intermediate member 109 includes a susbtantially U-shaped lower wire extending between the lower collars, the lower collars being provided laterally outwardly relative to the lower wire. Thus, the pair of lower element engaging portions 107 and the lower element intermediate member 109 cooperatively form a substantially U-shaped configuration. In some embodiments of the invention, each lower collar is made of a metal conventionally used in dental treatment and therapy.

Furthermore, in use, the upper element intermediate member 108 extends between the pair of upper element engaging portions 106 adjacently along an outer peripheral portion of the maxillary dental arch 420, at an intermediate vertical position relative thereto. Similarly, the lower element intermediate member 109 extends between the pair of lower element engaging portions 107 adjacently along an inner peripheral portion of the mandibular dental arch 422, at an intermediate vertical position relative thereto. In use, as seen in FIG. 6, the upper element intermediate member 108 is positioned between the maxillary component abutment surface 314 and the maxillary anterior teeth 414 and the lower element intermediate member 109 is positioned between the mandibular component abutment surface 318 and the mandibular anterior teeth 418. Thus, forces exerted by the maxillary and mandibular components 312 and 316 respectively on maxillary and mandibular dental arches 420 and 422 are distributed at least partially to the posterior lateral portions thereof, which reduces or eliminates the forces that could displace the anterior teeth.

In some embodiments of the invention, the intraoral device 100 further comprises at least one spacer 110. Typically, four spacers 110 are provided, but more or less spacers are within the scope of the appended claims. The spacers 110 are resiliently deformable and each mounted to one of the upper and lower wire for spacing apart the upper or lower wire from the posterior teeth along a posterior portion of the upper or lower wire. To that effect, the upper or lower wire extends through the spacer 110 and the latter abuts against one more of the maxillary or mandibular posterior teeth 424 and 426. Each spacer 110 has a suitable size and shape configuration such that no significant force is urged between the upper or lower element intermediate members 108 and 109 and the maxillary and mandibular posterior teeth 424 and 426.

In some embodiments of the invention, both the upper and lower elements 102 and 104 include each two spacer elements. Each one of the two spacers 110 is connected to a longitudinal portion of the upper and lower element intermediate members 108 and 109 extending from a position substantially adjacent a respective one of the upper or lower element engaging portions 106 or 107, to a position substantially adjacent the incisor teeth of the patient.

In some embodiments, the upper and lower wires are made of a substantially rigid metal wire used in dental treatment and therapy. Also, the spacers 110 are made of a suitable resilient polymeric material conventionally used in dental treatment and therapy. Examples of suitable materials include, but are not limited to, thermoplastic transparent heat-curing acrylic resins such as a polymethylmethacrylate (PMMA), rubber modified acrylics, vinyl acrylic copolymers, hydrophilic acrylics, a glycol-modified poly(ethylene terephthalate) such as that available under the trademark KODAR™ PETG copolyester 6763 (Eastman Chemical Products, Inc., Kingsport, Tenn.), and urethane polymers, such as the visible-light-curable urethane dimethacrylate polymer available under the trademark TRIAD™ (Dentsply International Inc., York, Pa.).

In an alternate embodiment, according to the present invention, there is provided an intraoral device comprising only one of the upper or lower elements 102 or 104 as the ones described in the first embodiment above. Thus, a patient having, for example, weakened teeth along only one of the dental arches may use the presently described embodiment. In another alternate embodiment (not shown in the figures), according to the present invention, there is provided an intraoral device that is substantially similar to the first embodiment 100 or the alternate embodiment described above. The difference in the presently described embodiment resides in that at least one of the upper or lower element 102 and 104 includes only one engaging portion 106. Furthermore, the upper or lower element intermediate member 108 or 109 extends from the only one engaging portion 106 to a position along the respective maxillary or mandibular dental arch 420 or 422 that is at least slightly passed the incisor teeth of the patient. Thus, a patient having no or is missing an appropriate adult tooth on one side of the dental arch may use the presently described embodiment.

In yet another alternate embodiment, according to the present invention, there is provided an intraoral device 200 as exemplified in FIG. 3. The intraoral device 200 is substantially similar in the manner in which it operates to the first and alternate embodiments described above in that it comprises an upper element 202 and a lower element 204. The upper element 202 and a lower element 204 each includes a pair of spaced apart engaging portions 206 and an intermediate member 208 extending between the engaging portions 206. Furthermore, the pair of engaging portions 206 and the intermediate member 208 of each one of the upper and lower elements 202 and 204 respectively, cooperatively form a substantially U-shaped configuration. Furthermore, each one in the pair of spaced apart engaging portions 206 of the upper element 202 has a substantially shell-like configuration.defining suitably adapted for removable engagement in a form-fit relationship with at least one tooth along a respective side of the maxillary dental arch 420. Furthermore, each one in the pair of spaced apart engaging portions 206 of the lower element 204 has a substantially shell-like configuration suitably adapted for removable engagement in a form-fit relationship with at least one tooth along a respective side of the mandibular dental arch 422. The upper and lower elements 202 and 204 therefore define a shell inner surface 205 conforming to an outer surface of at least part of the maxillary and mandibular dental arches 420 and 422.

Furthermore, the intermediate member 208 of the upper element 202 extends between the pair of engaging portions 206, adjacently along an outer peripheral portion of the mandibular dental arch 422, and at to intermediate vertical position relative thereof. Furthermore, the intermediate member 208 of the lower element 204 extends between the pair of engaging portions 206 adjacently along an inner peripheral portion 308 of the mandibular dental arch 422, and to an intermediate vertical position relative thereof. Still furthermore, each the intermediate member 208 of the upper and lower elements 202 and 204 respectively, has portions that extends substantially laterally on each side thereof to cover the teeth along which it is extending so as to form a shell-like configuration having a substantially U-shaped cross-section.

Thus, with the upper element 202 engaged on the maxillary dental arch 420, and the lower element 204 engaged on the mandibular dental arch 422, dental issues can be avoided, postponed or delayed in a patient using a mandibular advancement device 310 or the like which simultaneously apply a rearward force on a transversal front surface portion of the maxillary dental arch 420, and a forward force on a transversal rear surface portion of the mandibular dental arch 422, respectively.

In some embodiments of the presently described invention, each one of the upper and lower elements 202 and 204 respectively, is a single piece element formed from a suitable resilient polymeric material used in dental treatment and therapy, such as the ones described further above. In some other embodiments of the presently described invention, each one of the upper and lower elements 202 and 204, respectively, extends in a shell-like configuration having a substantially U-shaped cross-section, and has a suitable size and shape configuration to cover its respective entire maxillary and mandibular dental arches 420 and 422.

In yet some other embodiments of the presently described invention, each one of the upper and lower elements 202 and 204, respectively, have their respective U-shaped cross-section that extends further laterally so as to at least slightly overlap the gingivae, or gum, along the junction with the maxillary and mandibular dental arches 420 and 422.

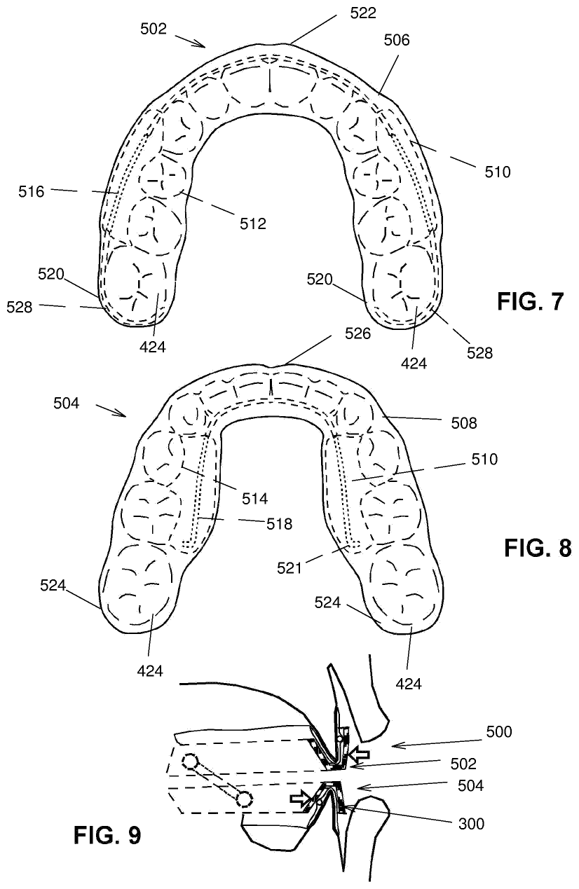

Referring to FIGS. 7 to 9, there is shown yet another intraoral device 500 that includes features from both the intraoral devices 100 and 200. Similarly to the intraoral device 200, the intraoral device 500 includes an upper shell 502, better seen for example in FIG. 7, and a lower shell 504, better seen for example in FIG. 8. The upper and lower shells 502 and 504 include respectively upper and lower shell bodies 506 and 508 made of a body material and inserts 510 made of an insert material provided inside the upper and lower shell bodies 506 and 508 and defining part of the upper and lower shell inner surfaces 512 and 514. Typically, the insert material is more rigid than the body material, allowing to better anchor relatively soft upper and lower shell bodies 506 and 508. The inserts 510 are positioned similarly to the spacers 110 when the intraoral device 500 is in use and are made of a similar material having similar properties as the spacers 110.

Typically, the body material is a polymer. To allow manufacturing upper and lower shells 502 and 504 that are relatively thin, and as such resiliently deformable to a greater extend than the intraoral device 200, the upper and lower shells 502 and 504 include respectively an upper reinforcing wire 516 and a lower reinforcing wire 518. Lowering the rigidity of the upper and lower shells 502 and 504 increases the comfort of the intraoral device 500 when worn by a patient.

The upper reinforcing wire 516 extends between the upper element engaging portions 520 and along the upper element intermediate member 522. The upper reinforcing wire 516 is typically metallic and coupled to the upper shell body 506 for mechanically reinforcing the upper shell body 506. For example, at least part of the upper reinforcing wire 516 is molded inside the upper shell body 506, or the upper reinforcing wire 516 is secured with an adhesive to the upper shell body 506. Typically, the upper reinforcing wire 516 extends through the inserts 510 of the upper shell 502.

The upper reinforcing wire 516 may have any suitable shape and position. For example, the upper reinforcing wire 516 is substantially U-shaped and terminated by a pair of hooks 528 in the upper element engaging portions 520. The hooks 528 are configured and sized for curving around a respective one of the maxillary posterior teeth 424. In other embodiments, the hooks 528 are omitted. Typically, the upper reinforcing wire 516 is configured to be laterally outward and anterior relative to the maxillary dental arch when the upper shell 502 is operationally mounted to the maxillary dental arch. That is, the upper reinforcing wire 516 is, in use, positioned similarly to the upper portion of the intraoral device 100.

Similarly, the lower reinforcing wire 518 extends between the lower element engaging portions 524 and along the lower element intermediate member 526. The lower reinforcing wire 518 is typically metallic and coupled to the lower shell body 508 for mechanically reinforcing the latter. For example, at least part of the lower reinforcing wire 518 is molded inside the lower shell body 508, or the lower reinforcing wire 518 is secured with an adhesive to the lower shell body 508. Typically, the lower reinforcing wire 518 extends through the inserts 510 of the lower shell 504.

The lower reinforcing wire 518 may have any suitable shape and position. For example, the lower reinforcing wire 518 is substantially U-shaped and terminated by a laterally outwardly extending segment 521 in each one of the lower element engaging portions 524, for example inside the inserts 510. In other embodiments, outwardly extending segments 521 are omitted. Typically, the lower reinforcing wire 518 is configured to be laterally inward and posterior relative to the mandibular dental arch when the lower shell 504 is operationally mounted to the mandibular dental arch. That is, the lower reinforcing wire 518 is, in use, positioned similarly to the lower portion of the intraoral device 100.

The inserts 510 may be molded integrally with or secured with an adhesive to the upper and lower shell bodies 506 and 508, or may be removable therefrom. The inserts 510 may be less rigid than, equally rigid with or more rigid than the upper and lower shell bodies 506 and 508.

A method of using the intraoral devices 100, 200 and 500 is now described, with specific reference to the intraoral device 100. The proposed method may prevent, delay or reduce teeth displacement in the mouth 410 of an intended user when using a mandibular advancement device 310. In some embodiments, and non-limitingly, cosmetic teeth displacements are prevented, delayed or reduced.

The method includes positioning an upper element intermediate member 108 adjacent a labial surface 412 of maxillary anterior teeth 414; anchoring the upper element intermediate member 108 to one or more maxillary posterior teeth 424; positioning the maxillary component 312 in the mouth with the maxillary component abutment surface 324 facing the labial surface 412 of the maxillary anterior teeth 414 so that the upper element intermediate member 108 is between at least part of the maxillary component abutment surface 324 and the maxillary anterior teeth 414. Similarly, the method includes positioning a lower element intermediate member 109 adjacent a lingual surface 416 of mandibular anterior teeth 418; anchoring the lower element intermediate member 109 to one or more mandibular posterior teeth 426; and positioning the mandibular component 316 in the mouth 410 with the mandibular component abutment surface 318 facing the lingual surface 416 of the mandibular anterior teeth 418 so that the lower element intermediate member 109 is between at least part of the mandibular component abutment surface 318 and the mandibular anterior teeth 418. These steps are followed by biasing the mandibular component 316 in an anterior direction relative to the maxillary component 312 and distributing at least partially anterior forces exerted by the mandibular component 316 and posterior forces exerted by the maxillary component 312 respectively to the mandibular and maxillary posterior teeth 426 and 424.

Using the intraoral device 100, one performs this method typically by positioning the lower wire along the mandibular anterior teeth 418 and inserting the lower collars around one or more of the mandibular posterior teeth 426, and similarly positioning the upper wire along the maxillary anterior teeth 414 and inserting the upper collars around one or more of the maxillary posterior teeth 424. Then, the maxillary and mandibular components 312 and 316 are positioned over the maxillary and mandibular dental arches 420 and 422 in a conventional manner, over the upper and lower elements 102 and 104. Typically, the maxillary and mandibular components 312 and 316 engage the upper and lower elements 102 and 104 in addition to or instead of engaging directly only the mandibular and maxillary anterior teeth 418 and 414.

When using the intraoral devices 200 or 500, one simply positions the upper and lower elements 202 and 204 over the maxillary and mandibular dental arches 420 and 422 and positions the mandibular device 310 over the intraoral device 200.

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention can thus be modified without departing from the spirit and nature of the subject invention as defined in the appended claims.

What is claimed is:

1. An intraoral device for preventing teeth displacement when using a mandibular advancement device, the mandibular advancement device including a maxillary component defining a maxillary component abutment surface for exerting a posterior force against a labial surface of maxillary anterior teeth, a mandibular component defining a mandibular component abutment surface for exerting an anterior force against a lingual surface of mandibular anterior teeth, and a link extending therebetween, the intraoral device comprising:
an upper element defining a pair of upper element engaging portions each configured for retentive engagement with a respective posterior lateral portion of a maxillary dental arch and an upper element intermediate member extending between the upper element engaging portions;
a lower element defining a pair of lower element engaging portions each configured for retentive engagement with a respective posterior lateral portion of a mandibular dental arch and a lower element intermediate member extending between the lower element engaging portions;
wherein, in use, the upper element intermediate member is positioned between the maxillary component abutment surface and the maxillary anterior teeth and the lower element intermediate member is positioned between the mandibular component abutment surface and the mandibular anterior teeth;
whereby forces exerted by the maxillary and mandibular components respectively on maxillary and mandibular dental arches are distributed at least partially to the posterior lateral portions thereof.

2. The intraoral device as defined in claim 1, wherein the upper element engaging portions each include a respective upper collar configured and sized for encircling one or more respective ones of the maxillary posterior teeth.

3. The intraoral device as defined in claim 2, wherein the upper element engaging portions each include a respective upper collar configured and sized for encircling a respective one of the maxillary posterior teeth.

4. The intraoral device as defined in claim 3, wherein the upper element intermediate member includes a susbtantially U-shaped upper wire extending between the upper collars, the upper collars being provided laterally inwardly relative to the upper wire.

5. The intraoral device as defined in claim 4, further including at least one resiliently deformable upper spacer mounted to the upper wire for spacing apart the upper wire from the maxillary posterior teeth along a posterior portion of the upper wire.

6. The intraoral device as defined in claim 1, wherein the lower element engaging portions each include a respective lower collar configured and sized for encircling one or more respective ones of the mandibular posterior teeth.

7. The intraoral device as defined in claim 6, wherein the lower element engaging portions each include a respective lower collar configured and sized for encircling a respective one of the mandibular posterior teeth.

8. The intraoral device as defined in claim 6, wherein the lower element intermediate member includes a susbtantially U-shaped lower wire extending between the lower collars, the lower collars being provided laterally outwardly relative to the lower wire.

9. The intraoral device as defined in claim 8, further including at least one resiliently deformable lower spacer mounted to the lower wire for spacing apart the lower wire from the mandibular posterior teeth along a posterior portion of the upper wire.

10. The intraoral device as defined in claim 1, wherein the upper element engaging portions and the upper element intermediate member are formed integrally in an upper shell defining an upper shell inner surface conforming to at least part of an outer surface of the maxillary dental arch.

11. The intraoral device as defined in claim 10, wherein the upper shell includes an upper shell body made of a body material and an insert made of an insert material provided inside the upper shell body and defining part of the upper shell inner surface, the insert material being more rigid than the body material.

12. The intraoral device as defined in claim 11, wherein the body material is a polymer and wherein the upper shell also includes an upper reinforcing wire extending between the upper element engaging portions and along the upper element intermediate member, the upper reinforcing wire being metallic and coupled to the upper shell body for mechanically reinforcing the upper shell body.

13. The intraoral device as defined in claim 12, wherein the upper reinforcing wire is terminated by a pair of hooks in the upper element engaging portion, the hooks being configured and sized for curving around a respective one of the maxillary posterior teeth.

14. The intraoral device as defined in claim 12, wherein the upper reinforcing wire extends through the insert.

15. The intraoral device as defined in claim 12, wherein the upper reinforcing wire is configured to be laterally outward and anterior relative to the maxillary dental arch when the upper shell is operationally mounted to the maxillary dental arch.

16. The intraoral device as defined in claim 1, wherein the lower element engaging portions and the lower element intermediate member are formed integrally in a lower shell defining a lower shell inner surface conforming to at least part of an outer surface of the mandibular dental arch.

17. The intraoral device as defined in claim 16, wherein the lower shell includes a lower shell body made of a body material and an insert made of an insert material provided inside the lower shell body and defining part of the lower shell inner surface, the insert material being more rigid than the body material.

18. The intraoral device as defined in claim 17, wherein the body material is a polymer and wherein the lower shell also includes a lower reinforcing wire extending between the lower element engaging portions and along the lower element intermediate member, the lower reinforcing wire being metallic and coupled to the lower shell body for mechanically reinforcing the lower shell body.

19. The intraoral device as defined in claim 18, wherein the lower reinforcing wire is terminated by a laterally outwardly extending segment in each one of the lower element engaging portions.

20. The intraoral device as defined in claim 18, wherein the lower reinforcing wire extends through the insert.

21. The intraoral device as defined in claim 18, wherein the lower reinforcing wire is configured to be laterally inwardly and posterior relative to the mandibular dental arch when the lower shell is operationally mounted to the mandicular dental arch.

22. A method for preventing, delaying or reducing teeth displacement in a mouth of an intended user when using a mandibular advancement device, the mandibular advancement device including a maxillary component defining a maxillary component abutment surface, a mandibular component defining a mandibular component abutment surface, and a link extending therebetween, the method comprising:
positioning an upper element intermediate member adjacent a labial surface of maxillary anterior teeth;
anchoring the upper element intermediate member to one or more maxillary posterior teeth;
positioning the maxillary component in the mouth with the maxillary component abutment surface facing the labial surface of the maxillary anterior teeth so that the upper element intermediate member is between at least part of the maxillary component abutment surface and the maxillary anterior teeth;
positioning a lower element intermediate member adjacent a lingual surface of mandibular anterior teeth;
anchoring the lower element intermediate member to one or more mandibular posterior teeth;
positioning the mandibular component in the mouth with the mandibular component abutment surface facing the lingual surface of the mandibular anterior teeth so that the lower element intermediate member is between at least part of the mandibular component abutment surface and the mandibular anterior teeth;
biasing the mandibular component in an anterior direction relative to the maxillary component;
distributing at least partially anterior forces exerted by the mandibular component and posterior forces exerted by the maxillary component respectively to the mandibular and maxillary posterior teeth.

23. The method as defined in claim 22, wherein upper and lower element intermediate members include respectively an upper wire and a lower wire, and wherein
positioning the mandibular component in the mouth so that the lower element intermediate member is between at least part of the mandibular component abutment surface and the mandibular anterior teeth includes positioning the mandibular component and the lower wire so that the lower wire is between the mandibular component abutment surface and the mandibular anterior teeth; and
positioning the maxillary component in the mouth so that the upper element intermediate member is between at least part of the maxillary component abutment surface and the maxillary anterior teeth includes positioning the maxillary component and the upper wire so that the upper wire between the maxillary component abutment surface and the maxillary anterior teeth.

24. The method as defined in claim 23, wherein
anchoring the upper element intermediate member to one or more maxillary posterior teeth includes inserting a pair of upper collars each extending at a respective end of the upper wire around the one or more of the maxillary posterior teeth located on a respective lateral side on which the upper collars are located; and
anchoring the lower element intermediate member to one or more mandibular posterior teeth includes inserting a pair of lower collars each extending at a respective end of the lower wire around the one or more of the mandibular posterior teeth located on a respective lateral side on which the lower collars are located.

25. The method as defined in claim 24, further comprising spacing apart the upper and lower wires from respectively at least some of the maxillary and mandibular posterior teeth with one or more resiliently deformable spacer positioned between the maxillary and mandibular posterior teeth and respectively the upper and lower wires.

26. The method as defined in claim 22, comprising covering at least some of the mandibular and maxillary teeth with respectively a lower shell and an upper shell and positioning the maxillary and mandibular components in the mouth over respectively the upper and lower shells.

27. An intraoral device for preventing teeth displacement when using a mandibular advancement device, the mandibular advancement device including a maxillary component defining a maxillary component abutment surface for exerting a posterior force against a labial surface of maxillary anterior teeth, a mandibular component defining a mandibular component abutment surface for exerting an anterior force against a lingual surface of mandibular anterior teeth, and a link extending therebetween, the intraoral device comprising:
an engaging portions configured for retentive engagement with a posterior lateral portion of a dental arch and an intermediate member extending from the engaging portion configured for conforming to an anterior portion of the dental arch
wherein, in use, the intermediate member is positioned between either the maxillary component abutment surface and the maxillary anterior teeth or between the mandibular component abutment surface and the mandibular anterior teeth;
whereby forces exerted by the maxillary and mandibular components respectively on one of the maxillary and mandibular dental arches are distributed at least partially to the posterior lateral portions thereof.
